(19) United States
(12) Patent Application Publication
(10) Pub. No.: US 2026/0096733 A1
(43) Pub. Date: Apr. 9, 2026

(54) WEARABLE SYSTEM AND BLOOD PRESSURE MEASUREMENT METHOD WHICH CAN BE PERFORMED BY THE WEARABLE SYSTEM

(71) Applicant: PixArt Imaging Inc., Hsin-Chu City (TW)

(72) Inventors: Shih-Jen Lu, Hsin-Chu City (TW); Chih-Hao Wang, Hsin-Chu City (TW); Chien-Yi Kao, Hsin-Chu City (TW); Yang-Ming Chou, Hsin-Chu City (TW); Hung-Chih Wang, Hsin-Chu City (TW); Hsin-Yi Lin, Hsin-Chu City (TW)

(73) Assignee: PixArt Imaging Inc., Hsin-Chu City (TW)

(21) Appl. No.: 18/909,923

(22) Filed: Oct. 8, 2024

(51) Int. Cl. A61B 5/021; A61B 5/00; A61B 5/022

(52) U.S. Cl. CPC A61B 5/02125; A61B 5/02116; A61B 5/02141; A61B 5/02233; A61B 5/6824; A61B 5/6826; A61B 5/6831; A61B 5/7221; A61B 5/7264; A61B 5/7271; A61B 2562/0247

(57) ABSTRACT

A wearable system comprising a first wearable device is disclosed. The first wearable device comprises: an enclosed outer ring; a first adjustable structure, configured to adjust an internal wearing space of the first wearable device while a length of the enclosed outer ring is fixed, wherein a user puts a first body portion thereof in the internal wearing space; a first light source, configured to emit first light toward the internal wearing space; a first PPG sensor, configured to detect a first PPG signal generated according to the first light; a first pressure sensor, configured to sense a pressure caused by the first body portion. A related blood pressure measurement method is also disclosed. Via the disclosed system and method, the blood pressure may be easily measured by wearable devices. Additionally, the wearable device does not need to be frequently calibrated based on the measurement of the sphygmomanometer.

### BACKGROUND OF THE INVENTION

### 1. Field of the Invention

The present invention relates to a wearable system and a blood pressure measurement method, and particularly relates to a wearable system and a blood pressure measurement method which can measure blood pressure in an optical manner.

### 2. Description of the Prior Art

A conventional blood pressure measurement method always requires a specific device, such as a sphygmomanometer or a stethoscope. With the advancement of technology, wearable devices which can measure blood pressure are becoming more and more popular. However, such blood pressure measurement method is an indirect measurement method. For example, the blood pressure is measured based on PTT (Pulse transit time). Thus, when the user's blood pressure is unstable, the measured blood pressure may easily become inaccurate. Additionally, the wearable device which performs such blood pressure measurement method needs to be frequently calibrated based on the measurement of the sphygmomanometer.

Accordingly, a new wearable system and a new blood pressure measurement method are needed.

### SUMMARY OF THE INVENTION

One objective of the present invention is to provide a wearable system which can easily measure a blood pressure.

Another objective of the present invention is to provide a wearable system which can easily measure a blood pressure.

One embodiment of the present invention is to provide a wearable system, comprising: a first wearable device, comprising: an enclosed outer ring; a first adjustable structure, attached to the enclosed outer ring, configured to adjust an internal wearing space of the first wearable device while a length of the enclosed outer ring is fixed, wherein a user puts a first body portion thereof in the internal wearing space while wearing the first wearable device; a first light source, configured to emit first light toward the internal wearing space; a first PPG (Photoplethysmography) sensor, configured to detect a first PPG signal generated according to the first light; a first pressure sensor, configured to sense a pressure caused by the first body portion.

Another embodiment of the present invention discloses a blood pressure measurement method, applied to a first wearable device with an enclosed outer ring, comprising: (a) emitting first light toward an internal wearing space of a first wearable device, wherein the internal wearing space is adjustable while a length of the enclosed outer ring is fixed, wherein a user puts a first body portion thereof in the internal wearing space while wearing the first wearable device; (b) detecting a first PPG signal generated according to first light emitted by a first light source of the first wearable device, by a first PPG sensor of the first wearable device; (c) sensing a pressure caused by the first body portion by a first pressure sensor of the first wearable device; and (d) computing a blood pressure of the user according to the pressure and a signal integrity of the PPG signal.

In view of above-mentioned embodiment, the blood pressure may be easily measured by wearable devices. Additionally, the wearable device which performs such blood pressure measurement method does not need to be frequently calibrated based on the measurement of the sphygmomanometer.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a schematic diagram illustrating a wearable system according to one embodiment of the present invention.

FIG. 2 is a schematic diagram illustrating the states of “opened” and “enclosed” of the enclosed outer ring in FIG. 1, according to one embodiment of the present invention.

FIG. 3A and FIG. 3B are schematic diagrams illustrating wearable systems according to embodiments of the present invention.

FIG. 4 is a schematic diagram illustrating operations of the wearable systems in FIG. 1, FIG. 3A and FIG. 3B, according to one embodiment of the present invention.

FIG. 5 is a schematic diagram illustrating a wearable system according to another embodiment of the present invention.

FIG. 6 is a schematic diagram illustrating a detail structure of the second wearable device, according to one embodiment of the present invention.

FIG. 7 and FIG. 8 are schematic diagrams illustrating operations of the wearable system in FIG. 5 and FIG. 6, according to embodiments of the present invention.

FIG. 9 is a flow chart illustrating a blood pressure measurement method according to one embodiment of the present invention.

FIG. 10 is a flow chart illustrating a blood pressure measurement method according to another embodiment of the present invention.

### DETAILED DESCRIPTION

In the following descriptions, several embodiments are provided to explain the concept of the present application. The term “first”, “second”, “third” in following descriptions are only for the purpose of distinguishing different one elements, and do not mean the sequence of the elements. For example, a first device and a second device only mean these devices can have the same structure but are different devices.

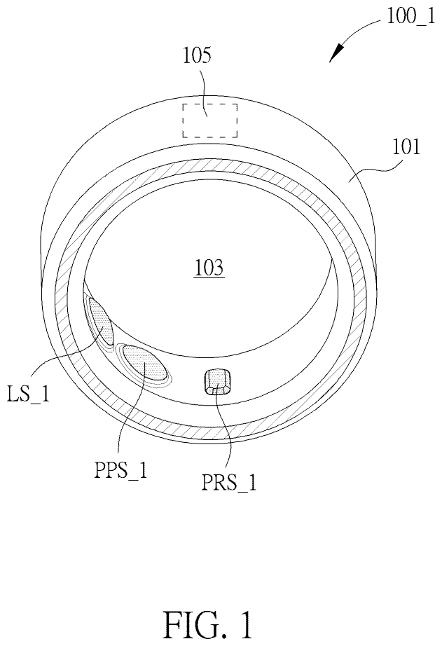

FIG. 1 is a schematic diagram illustrating a wearable system according to one embodiment of the present invention. In the embodiment of FIG. 1, the wearable system comprises a first wearable device 100_1, which is a ring, but the first wearable device 100_1 may be any other device besides the ring. As shown in FIG. 1, the first wearable device 100_1 comprises a first adjustable structure, an enclosed outer ring 101, a first light source LS_1, a first PPG sensor PPS_1, and a first pressure sensor PRS_1. Details of the first adjustable structure will be described later. In one embodiment, the enclosed outer ring 101 may be made of solid, such as wood, plastic, ceramics or metal. In such case, the enclosed outer ring 101 has a fixed shape and a fixed length.

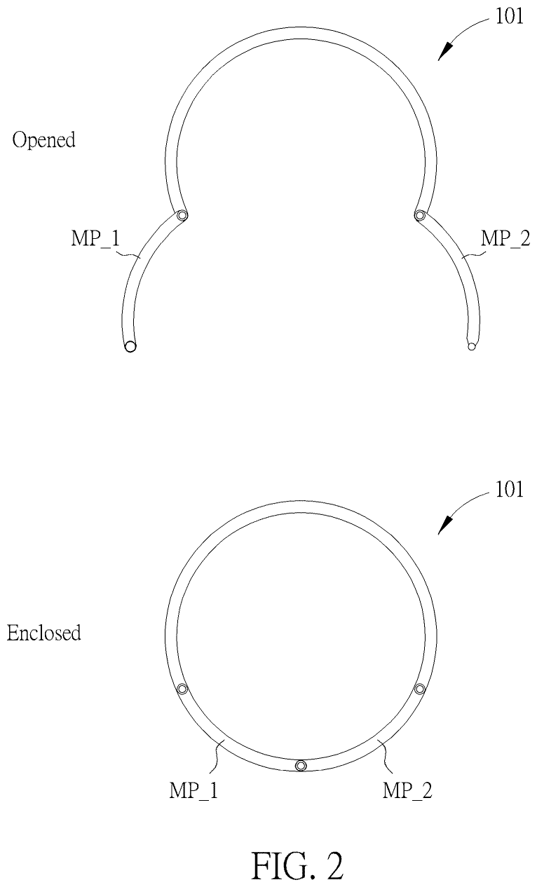

In one embodiment, the enclosed outer ring 101 can be opened or enclosed. FIG. 2 is a schematic diagram illustrating the states of “opened” and “enclosed” of the enclosed outer ring 101 in FIG. 1, according to one embodiment of the present invention. Please note the enclosed outer ring 101 may be “opened” or “enclosed” via different structures rather than limited to the structure shown in FIG. 2.

As shown in FIG. 2, the enclosed outer ring 101 comprises movable portions MP_1 and MP_2. The movable portions MP_1 and MP_2 are in opened locations in the upper diagram of FIG. 2, thus the enclosed outer ring 101 is opened (does not have an enclosed shape). On the contrary, in the lower diagram of FIG. 2, the movable portions MP_1 and MP_2 are in enclosed locations, thus the enclosed outer ring 101 is enclosed (has an enclosed shape). Please note, no matter the enclosed outer ring 101 is “opened” or “enclosed”, other components of the first wearable device 100_1 may still be attached to the enclosed outer ring 101. In one embodiment, the enclosed outer ring 101 is opened when the first wearable device 100_1 is not worn by a user or to be worn by the user. On the opposite, the enclosed outer ring 101 is enclosed when the first wearable device 100_1 is already worn by the user.

The first adjustable structure is attached to the enclosed outer ring, configured to adjust an internal wearing space 103 of the first wearable device 100_1 while a length of the enclosed outer ring is fixed. In other words, a length of the enclosed outer ring 101 is a first length when the internal wearing space 103 of the first wearable device 100_1 is a first value, but the length of the enclosed outer ring 101 is still the first length when the internal wearing space 103 of the first wearable device 100_1 is adjusted from a first value to a second value. A user puts a first body portion thereof in the internal wearing space 103 while wearing the first wearable device 100_1. For example, a user puts a finger thereof in the internal wearing space 103 while wearing the first wearable device 100_1, if the first wearable device 100_1 is a ring. For another example, a user puts a wrist thereof in the internal wearing space 103 while wearing the first wearable device 100_1, if the first wearable device 100_1 is a wristband. For still another example, a user puts an upper arm thereof in the internal wearing space 103 while wearing the first wearable device 100_1, if the first wearable device 100_1 is an armband.

In one embodiment, the length of the enclosed outer ring 101 can be adjusted in a non-operation mode, but the length of the enclosed outer ring 101 cannot be adjusted in an operation mode. The operation mode may mean, for example, the first wearable device 100_1 is computing the blood pressure of the user or will compute the blood pressure of the user. On the opposite, the non-operation mode may mean the first wearable device 100_1 is not computing the blood pressure. However, the operation mode and the on-operation mode may have different meanings.

Accordingly, in the operation mode, the internal wearing space 103 can be changed by the first adjustable structure rather than changed by varying the length of the enclosed outer ring 101. The function of” the length of the enclosed outer ring 101 cannot be adjusted in the operation mode” may be implemented by various methods. For example, if the length of the enclosed outer ring 101 can be changed by executing a program, the program may be designed to that the user could not change the length of the enclosed outer ring 101 in the operation mode.

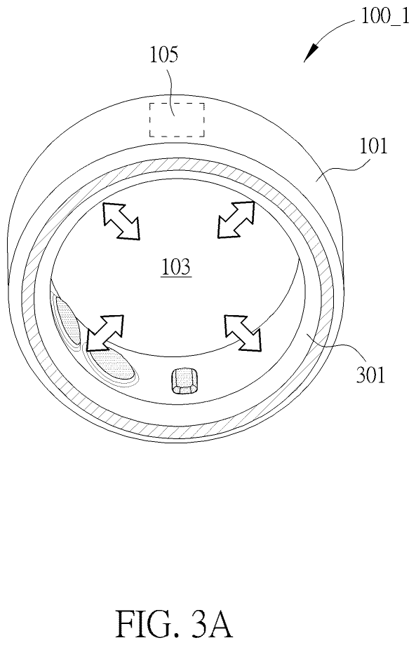

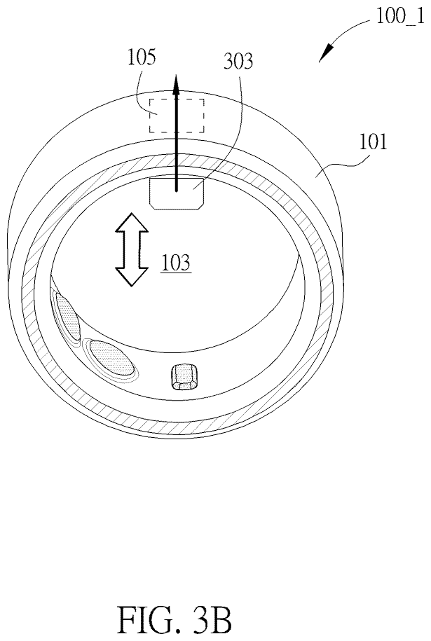

The first adjustable structure may be various kinds of structures. FIG. 3A and FIG. 3B are schematic diagrams illustrating wearable systems according to embodiments of the present invention. Please note, for the convenience of explaining, some symbols of the components are not illustrated in FIG. 3A and FIG. 3B. In the embodiment of FIG. 3A, the first adjustable structure is an inflatable cuff 301. When the inflatable cuff 301 is inflated, the internal wearing space 103 becomes smaller. On the opposite, when the inflatable cuff 301 is not inflated, the internal wearing space 103 becomes larger.

In the embodiment of FIG. 3B, the first adjustable structure is a dynamic button 303, which can be pressed or released. When the dynamic button 303 is released (e.g., released to be inside the enclosed outer ring 101 of the first wearable device 100_1), the internal wearing space 103 becomes larger thus a smaller pressure is provided to the user. On the contrary, when the dynamic button 303 is pressed (e.g., pressed to protrude outside the enclosed outer ring 101 of the first wearable device 100_1), the internal wearing space 103 becomes smaller thus a larger pressure is provided to the user. Please note, in one embodiment, the dynamic button 303 is gradually released in a predetermined time interval after the dynamic button 303 is pressed. First adjustable structure with other structures can also follow such operations. Please note, the structures of the first adjustable structure are not limited to the examples illustrated in FIG. 3A and FIG. 3B. For example, the first adjustable structure can be a mechanical structure.

Please refer to FIG. 1 again. The first light source LS_1 is configured to emit first light toward the internal wearing space 103. The first PPG sensor PPS_1, which may be an optical sensor such as an image sensor or a photo detector, is configured to detect a first PPG signal generated according to the first light. The first pressure sensor PRS_1 is configured to sense a pressure caused by the above-mentioned first body portion in the internal wearing space 103.

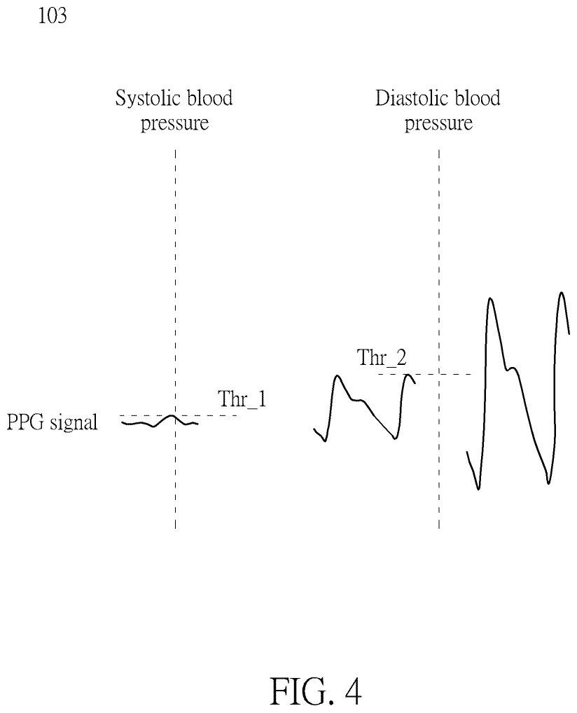

The first wearable device 100_1 may further comprise a processing circuit 105, which is configured to compute a blood pressure of the user according to the pressure sensed by the first pressure sensor PRS_1, and a signal integrity of the PPG signal detected by the first PPG sensor PPS_1 (e.g., the first PPG signal). FIG. 4 is a schematic diagram illustrating operations of the wearable systems in FIG. 1, FIG. 3A and FIG. 3B, according to one embodiment of the present invention. As shown in FIG. 4, if the internal wearing space 103 is small, the user's blood vessels will be compressed, thus the signal integrity of the PPG signal is low (i.e., the waveform of the PPG signal is incomplete). In such case, the processing circuit 105 may compute a systolic blood pressure according to the pressure when the signal integrity is increasing to be larger than a first integrity level Thr_1. The signal integrity can be replaced by an amplitude of the PPG signal. In one embodiment, the systolic blood pressure is M×pressure, M is a natural number. M can be determined by various methods. For example, a real systolic blood pressure is measured by a sphygmomanometer and then M can be determined by computing the relation between the systolic blood pressure and the pressure.

On the contrary, if the internal wearing space 103 is large, the user's blood vessels is not compressed, thus the signal integrity of the PPG signal is high (i.e., the waveform of the PPG signal is complete). In such case, the processing circuit 105 may compute a diastolic blood pressure according to the pressure if the signal integrity is increasing larger than a second integrity level Thr_2 which is higher than the first integrity level Thr_1. In one embodiment, the diastolic blood pressure is N×pressure, N is a natural number. N can be determined by various methods. For example, a real diastolic blood pressure is measured by a sphygmomanometer and then N can be determined by computing the relation between the real diastolic blood pressure and the pressure.

For more detail, the operation shown in FIG. 4 is a continuous process. The body portion of the user is pressed first (i.e., the internal wearing space 103 becomes small), to make the waveform of the PPG signal to become incomplete or disappears. After that, the body portion of the user is gradually released (i.e., the internal wearing space 103 gradually becomes large), to make the waveform of the PPG signal to gradually becomes complete. As above-mentioned, the above-mentioned systolic blood pressure and the diastolic blood pressure may be measured in such process.

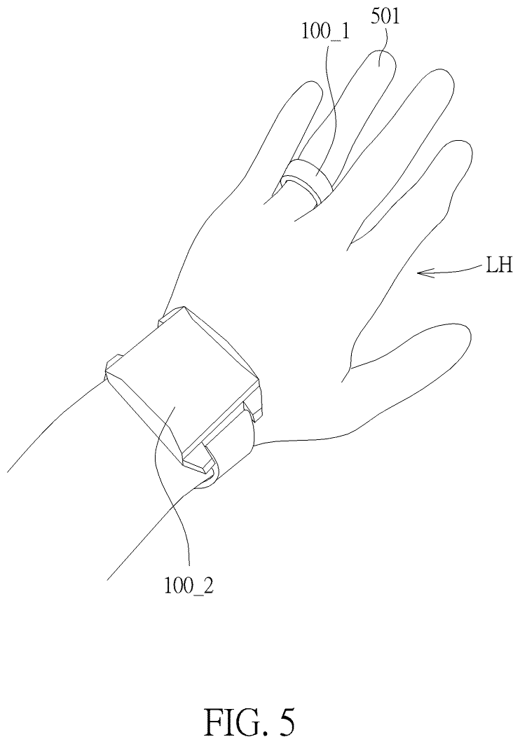

In the embodiment of FIG. 4, the blood pressure is measured based on the PPG signal. However, the blood pressure may be acquired by using the pressure sensor PRS_1 but not according to the PPG signal. Details of such embodiment will be described for more detail in following embodiments. Please note, either the blood pressure measurement using the PPG signal or the blood pressure measurement using the pressure sensor may also refer to a heart rate of the user to compute the systolic blood pressure or the diastolic blood pressure. In above-mentioned embodiments, the wearable system comprises only one wearable device (the first wearable device 100_1). However, the wearable system may comprise more than one wearable device. FIG. 5 is a schematic diagram illustrating a wearable system according to another embodiment of the present invention. As shown in FIG. 5, the wearable system further comprises a second wearable device 100 2 besides the first wearable device 100_1. In the embodiment shown in FIG. 5, the first wearable device 100_1 is a ring worn on a finger 501 of a left hand LH, and the second wearable device 100 2 is a watch worn on a wrist of the left hand LH. Accordingly, a distance between the first wearable device 100_1 and a heart of the user is larger than a distance between the second wearable device 100 2 and the heart, while the user wearing the first wearable device 100_1 and the second wearable device 100 2. In other words, the second wearable device 100 2 is closer to a heart of the user than the first wearable device 100_1. The first wearable device 100_1 and the second wearable device 100 2 may be replaced by other wearable devices rather than limited to the ring and the watch.

Briefly, in one embodiment, two wearable devices are worn on the same arm of a user. If the wearable device which is closer to the heart presses the arm, the blood pressure measured by the wearable device which is farer from the heart will be affected. Detail operations of the wearable devices are described in following descriptions.

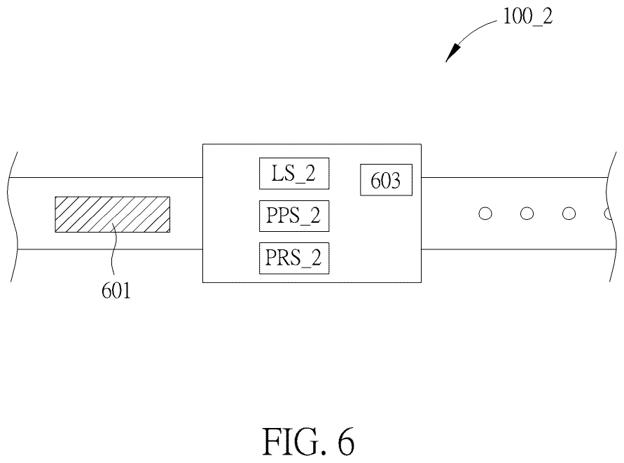

The first wearable device 100_1 may comprise the structures illustrated in the embodiments of FIG. 1-FIG. 5, but not limited. FIG. 6 is a schematic diagram illustrating a detail structure of the second wearable device, according to one embodiment of the present invention. As shown in FIG. 6, the second wearable device 100_2 comprises a compression device 601, a processing circuit 603, a second pressure sensor PRS 2, a second PPG sensor PPS 2, and a second light source LS_2. Please note, the locations of components of the second wearable device 100_2 are not limited to the examples shown in FIG. 6. Besides, the second wearable device 100_2 may comprise only portion of the components show in FIG. 6.

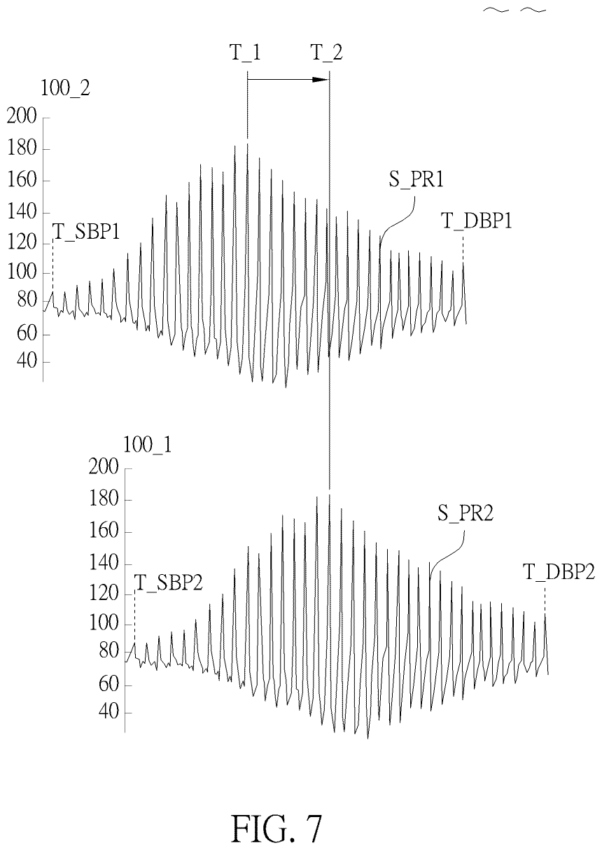

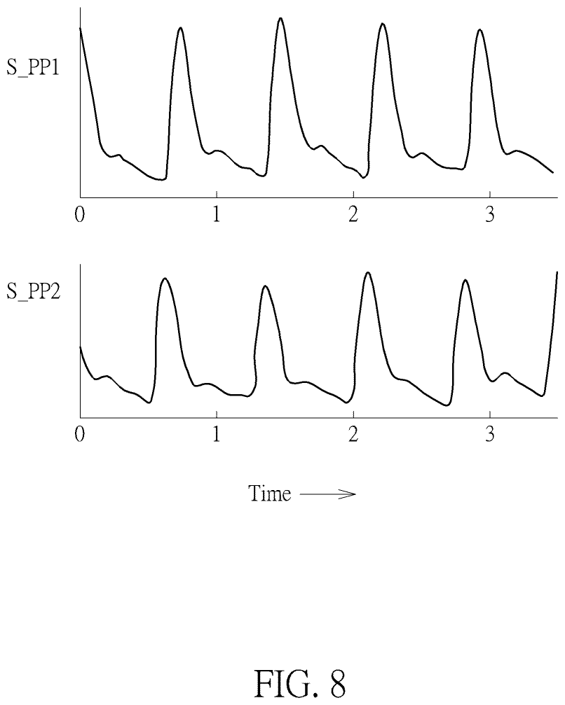

FIG. 7 and FIG. 8 are schematic diagrams illustrating operations of the wearable system in FIG. 5 and FIG. 6, according to embodiments of the present invention. In the embodiment of FIG. 7, the compression device 601 compresses and then gradually releases a second body portion of the user in a predetermined time interval, thereby the blood vessels located in the second body portion are compressed and then released. The processing circuit 603 computes a time difference between a first arriving time T_1 of a first pressure signal S_PR1 and a second arriving time T_2 of a second pressure signal S_PR2 after the compression device 601 compresses the second body portion and then releases the second body portion.

Specifically, the first arriving time T_1 means a time that a peak region of the first pressure signal S_PR1 reaches the second pressure sensor PRS_2 of the second wearable device 100_2. Further, the second arriving time T_2 means a time that a peak region of the second pressure signal S_PR2 reaches the first pressure sensor PRS_1 of the first wearable device 100_1. The first pressure signal S_PR1 and the second pressure signal S_PR2 may both be caused by the compressing and releasing of the compression device 601. The processing circuit 603 of the second wearable device 100_2 computes a blood pressure of the user according to the time difference. Such method may also be regarded as computing a blood pressure of the user according to a pressure PPT signal. Please note, the operation of “computing a blood pressure according to the time difference” may also be performed by the processing circuit 105 of the first wearable device 100_1.

In one embodiment, a first signal start time T_SBP1 of the first pressure signal S_PR1 and a second signal start time T_SBP2 of the second pressure signal S_PR2 are acquired. Also, a first signal end time T_DBP1 of the first pressure signal S_PR1 and a second signal end time T_DBP2 of the second pressure signal S_PR2 are acquired. The signal start time may mean a time at which an amplitude of the pressure signal is increasing to be over a first signal threshold, and the signal end time may mean a time at which an amplitude of the pressure signal is decreasing to be lower than a second signal threshold. Besides, the heart rate of the user is also acquired. In such case, the blood pressure may be computed according to the time difference T1−T2, the first signal start time T_SBP1, the second signal start time T_SBP2, the first signal end time T_DBP1, the second signal end time T_DBP2, the heart rate, and peak values of the first pressure signal S_PR1 and a second signal end time T_DBP2. More specifically, the diastolic blood pressure may be computed according to the first signal end time T_DBP1, the second signal end time T_DBP2, the peak values and the heart rate. Besides, the systolic blood pressure may be computed according to the first signal start time T_SBP1, the second signal start time T_SBP2, the peak values and the heart rate. The heart rate mentioned here may be a mean heart rate in a predetermined time interval

As stated in the embodiment of FIG. 4, the blood pressure can be measured only according to the pressure signals sensed by the first pressure sensor PRS_1 and not according to the PPG signals. Such method is similar with the operations stated in FIG. 7. However, such embodiment has only one wearable device (the first wearable device 100_1). Accordingly, the time difference T1−T2, the first signal start time T_SBP1 and the first signal end time T_DBP1 cannot be acquired. Therefore, in such embodiment, after the first body portion of the user is pressed and then gradually released, the blood pressure may be computed according to the second signal start time T_SBP2, the second signal end time T_DBP2, the heart rate, and a peak value of the second pressure signal S_PR2. More specifically, the diastolic blood pressure may be computed according to the second signal end time T_DBP2, the peak value and the heart rate. Besides, the systolic blood pressure may be computed according to the second signal start time T_SBP2, the peak value and the heart rate. The heart rate mentioned here may be a mean heart rate in a predetermined time interval

In the embodiment of FIG. 8, the compression device 601 compresses and then releases a second body portion of the user, thereby the blood vessels located in the second body portion are compressed and then released, thereby the pressure signals shown in FIG. 7 can be acquired. The pressure signals can also be used to compute the blood pressure in the embodiment of FIG. 8, which will be described for more detail later.

Further, in the embodiment of FIG. 8, PPG signals can be acquired. For more detail, the second light source LS_2 emits second light. Also, the second PPG sensor PPS_2 detects a second PPG signal S_PP2 generated according to the second light. The processing circuit 603 computes a blood pressure of the user according PPT signals of the first PPG signal S_PP1 and the second PPG signal SPP_2. The first PPG signal S_PP1 is generated by the first PPG sensor PPS_1 of the first wearable device 100_1. Please note, the operation of “computing a blood pressure of the user according PPT signals” may also be performed by the processing circuit 105 of the first wearable device 100_1.

The embodiment which uses only the PPG signal without using the pressure signal, may be used in some situations where it is not possible to put pressure on the user or where it is not suitable to put pressure on the user (e.g., while the user is sleeping). For example, in one embodiment, the blood pressure is computed according to the PPG signal, the heart rate but not according to the pressure signal. In such case, the variation of the computed blood pressures may be used to monitor the user's physical conditions.

The embodiments shown in FIG. 7 and FIG. 8 can be combined. That is, the blood pressure is computed according to the PPG signals and the pressure signals. For example, the blood pressure may be computed according to the time difference T_1−T_2 (i.e., PPT signal), signal start times, signal end times, the heart rate, and peak values of pressure signals in FIG. 8, and according to PPT signals of the PPG signals. More specifically, the diastolic blood pressure may be computed according to the time difference T_1−T_2, the first signal end time T_DBP1, the second signal end time T_DBP2, the peak values, the heart rate and according to PPT signals of the PPG signals. Besides, the systolic blood pressure may be computed according to the time difference T_1−T2, the first signal start time T_SBP1, the second signal start time T_SBP2, the peak values, the heart rate and according to PPT signals of the PPG signals.

In one embodiment, the systolic blood pressure SBP and the diastolic blood pressure DBP can be computed according to the following Equation (1) and Equation (2):

$$SBP = A 1 \times PPG\_PTT + A 2 \times Pressure\_PTT + A 3 \times mean HR + A 3 \times HRV + C 1 \qquad Equation ( 1 )$$

$$DPG = B 1 \times PPG\_PTT + B 2 \times Pressure\_PTT + B 3 \times mean HR + B 3 \times HRV + C 2 \qquad Equation ( 2 )$$

PPG_PTT and Pressure_PTT are respectively PPT signals of PPG signals and pressure signals. Mean HR is a mean heart rate in a predetermined time interval. HRV is the variation rate of the heart rate. A1, A2, A3, B1, B2, C1 and C2 are constant values which can be set corresponding to different requirements or different designs of the wearable devices.

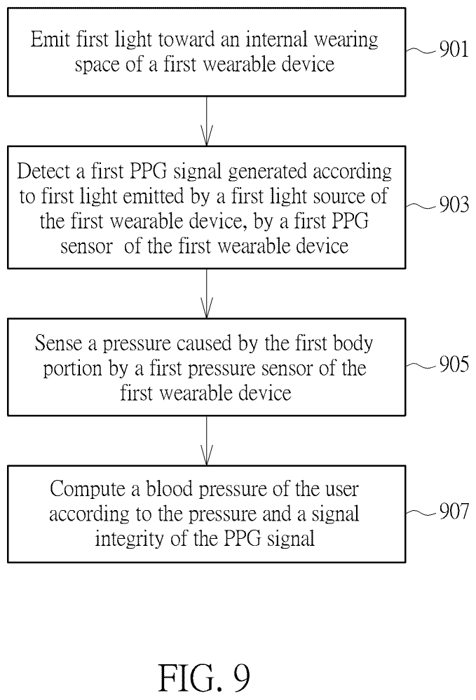

FIG. 9 is a flow chart illustrating a blood pressure measurement method according to one embodiment of the present invention. The blood pressure measurement method is applied to a first wearable device with an enclosed outer ring and comprises following steps:

### Step 901

Emit first light toward an internal wearing space of a first wearable device (e.g., first wearable device 100_1 in FIG. 1), wherein the internal wearing space is adjustable while a length of the enclosed outer ring (e.g., enclosed outer ring 103 in FIG. 1) is fixed, wherein a user puts a first body portion thereof in the internal wearing space while wearing the first wearable device.

### Step 903

Detect a first PPG signal generated according to first light emitted by a first light source of the first wearable device, by a first PPG sensor (e.g., first PPG sensor PPS_1 in FIG. 1) of the first wearable device.

### Step 905

Sense a pressure caused by the first body portion by a first pressure sensor (e.g., first pressure sensor PRS_1 in FIG. 1) of the first wearable device.

### Step 907

Compute a blood pressure of the user according to the pressure and a signal integrity of the PPG signal (e.g., the embodiment shown in FIG. 4).

Please note, in above-mentioned steps 901, 903, 905 and 907, the operations of detecting a PPG signal and sensing a pressure are performed by the same device (the first wearable device). However, these operations may be performed by different devices. For example, if the wearable system shown in FIG. 5 is used to compute a blood pressure following the concepts disclosed in steps 901, 903, 905 and 907. The operation of sensing a pressure may be performed by the second wearable device 100_2 and the step of detecting a PPG signal may be still performed by the first wearable device 100_1.

As above-mentioned, the blood pressure may be computed according to the pressure signal but not according to the PPF signal. In such case, the blood pressure measurement method in FIG. 9 may further comprise: sensing the pressure to generate a pressure sensing signal by the first pressure sensor; computing a blood pressure according to a signal start time of the pressure signal, a signal end time of the pressure signal, a heart rate of the user, and a peak value of the pressure signal.

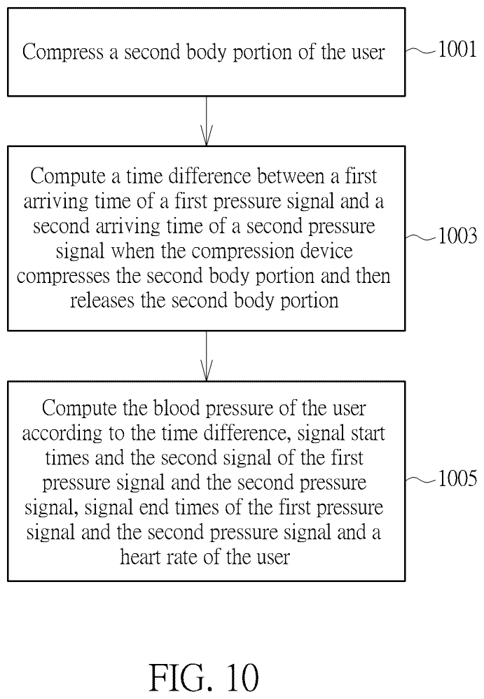

FIG. 10 is a flow chart illustrating a blood pressure measurement method according to another embodiment of the present invention. The blood pressure measurement method illustrated in FIG. 10 corresponding to a wearable system comprising a first wearable device and a second wearable device, such as the wearable system shown in FIG. 5. The first wearable device, which is worn on a first body portion of a user, comprises a first pressure sensor. Also, the second wearable device comprises a compression device and a second pressure sensor. In one embodiment, a distance between the first wearable device (e.g., the first wearable device 100_1) and a heart of the user is larger than a distance between the second wearable device (e.g., the second wearable device 100_2) and the heart, while the user wearing the first wearable device and the second wearable device.

The blood pressure measurement method in FIG. 10 comprises

Step 1001 Compressing (press) a second body portion of the user.

### Step 1003

Computing a time difference (e.g., the time difference T_1−T_2 in FIG. 7) between a first arriving time of a first pressure signal and a second arriving time of a second pressure signal when the compression device compresses the second body portion and then releases the second body portion.

The first arriving time means a time that a peak region of the first pressure signal reaches the first pressure sensor and the second arriving time means a time that a peak region of the second pressure signal reaches the second pressure sensor.

### Step 1005

Computing the blood pressure of the user according to the time difference, signal start times (e.g., the first signal start time T_SBP1 and the second signal start time T_SBP2) of the first pressure signal and the second pressure signal, signal end times (e.g., the first signal end time T_DBP1 and the second signal end time T_DBP2) of the first pressure signal and the second pressure signal and a heart rate of the user. As stated in the embodiment of FIG. 8, the blood pressure can further be computed according to PPT signals of PPG signals.

It will be appreciated the above-mentioned methods and systems can be used to measure other physiological parameters rather than limited to blood pressures.

In view of above-mentioned embodiment, the blood pressure may be easily measured by wearable devices. Additionally, the wearable device which performs such blood pressure measurement method does not need to be frequently calibrated based on the measurement of the sphygmomanometer.

Those skilled in the art will readily observe that numerous modifications and alterations of the device and method may be made while retaining the teachings of the invention. Accordingly, the above disclosure should be construed as limited only by the metes and bounds of the appended claims.

1. A wearable system, comprising:
a first wearable device, comprising:
an enclosed outer ring;
a first adjustable structure, attached to the enclosed outer ring, configured to adjust an internal wearing space of the first wearable device while a length of the enclosed outer ring is fixed, wherein a user puts a first body portion thereof in the internal wearing space while wearing the first wearable device;
a first light source, configured to emit first light toward the internal wearing space;
a first PPG (Photoplethysmography) sensor, configured to detect a first PPG signal generated according to the first light;
a first pressure sensor, configured to sense a pressure caused by the first body portion.

2. The wearable system of claim 1, wherein the enclosed outer ring can be opened or enclosed, wherein the enclosed outer ring is enclosed when the first wearable device is worn by the user.

3. The wearable system of claim 1, wherein the first wearable device further comprises:
a processing circuit, configured to compute a blood pressure of the user according to the pressure and a signal integrity of the PPG signal.

4. The wearable system of claim 3, wherein the processing circuit computes a systolic blood pressure according to the pressure when the signal integrity is increasing to be larger than a first integrity level.

5. The wearable system of claim 3, wherein the processing circuit computes a diastolic blood pressure according to the pressure when the signal integrity is increasing to be larger than a second integrity level.

6. The wearable system of claim 1,
wherein the first pressure sensor senses the pressure to generate a pressure sensing signal;
wherein the first wearable device further comprises:
a processing circuit, configured to compute a blood pressure according to a signal start time of the pressure signal, a signal end time of the pressure signal, a heart rate of the user, and a peak value of the pressure signal.

7. The wearable system of claim 1, wherein the first adjustable structure is an inflatable cuff or a dynamic button.

8. The wearable system of claim 1, wherein the first wearable device is a ring, a wristband, or an armband.

9. A wearable system, comprising:
a first wearable device, worn on a first body portion of a user, comprising:
a first pressure sensor;
a second wearable device, comprising:
a compression device, configured to compress a second body portion of the user; and
a second pressure sensor;
and
a processing circuit, configured to compute a time difference between a first arriving time of a first pressure signal and a second arriving time of a second pressure signal when the compression device compresses the second body portion and then releases the second body portion, wherein the first arriving time means a time that a peak region of the first pressure signal reaches the first pressure sensor and the second arriving time means a time that a peak region of the second pressure signal reaches the second pressure sensor;
wherein the processing circuit computes the blood pressure of the user according to the time difference, signal start times of the first pressure signal and the second pressure signal, signal end times of the first pressure signal and the second pressure signal and a heart rate of the user.

10. The wearable system of claim 9, wherein a distance between the first wearable device and a heart of the user is larger than a distance between the second wearable device and the heart, while the user wearing the first wearable device and the second wearable device.

11. A blood pressure measurement method, applied to a first wearable device with an enclosed outer ring, comprising:
(a) emitting first light toward an internal wearing space of the first wearable device by a first light source of the first wearable device, wherein the internal wearing space is adjustable while a length of the enclosed outer ring is fixed, wherein a user puts a first body portion thereof in the internal wearing space while wearing the first wearable device;
(b) detecting a first PPG signal generated according to the first light, by a first PPG sensor of the first wearable device;
(c) sensing a pressure caused by the first body portion by a first pressure sensor of the first wearable device; and
(d) computing a blood pressure of the user according to the pressure and a signal integrity of the first PPG signal.

12. The blood pressure measurement method of claim 11, wherein the enclosed outer ring can be opened or enclosed, wherein the enclosed outer ring is enclosed when the first wearable device is worn by the user.

13. The blood pressure measurement method of claim 11, wherein the step (d) computes a systolic blood pressure according to the pressure if the signal integrity is increasing to be larger than a first integrity level.

14. The blood pressure measurement method of claim 11, wherein the step (d) computes a diastolic blood pressure according to the pressure if the signal integrity is increasing to be larger than a second integrity level.

15. The blood pressure measurement method of claim 11, further comprising:
sensing the pressure to generate a pressure sensing signal by the first pressure sensor;
computing a blood pressure according to a signal start time of the pressure signal, a signal end time of the pressure signal, a heart rate of the user, and a peak value of the pressure signal.

16. The blood pressure measurement method of claim 11, wherein the first wearable device comprises a first adjustable structure, wherein the first adjustable structure is an inflatable cuff or a dynamic button.

17. The blood pressure measurement method of claim 11, further comprising:
computing a time difference between a first arriving time of a first pressure signal and a second arriving time of a second pressure signal when a compression device of a second wearable device compresses a second body portion of the user and then releases the second body portion, wherein the first arriving time means a time that a peak region of the first pressure signal reaches the first pressure sensor and the second arriving time means a time that a peak region of the second pressure signal reaches a second pressure sensor of the second wearable device; and
computing the blood pressure of the user according to the time difference.

18. The blood pressure measurement method of claim 17, wherein a distance between the first wearable device and a heart of the user is larger than a distance between the second wearable device and the heart, while the user wearing the first wearable device and the second wearable device.
